(19) United States
(12) Patent Application Publication
(10) Pub. No.: US 2024/0139485 A1
(43) Pub. Date: May 2, 2024

(54) MICRONEEDLE ARRAY WITH AN INTERLOCKING FEATURE

(71) Applicant: WASHINGTON STATE UNIVERSITY, Pullman, WA (US)

(72) Inventors: Kuen-Ren CHEN, Pullman, WA (US); Maher HN AMER, Pittsburgh, PA (US)

(21) Appl. No.: 18/547,903

(22) PCT Filed: Mar. 3, 2022
(86) PCT No.: PCT/US2022/018647

§ 371 Date: Aug. 25, 2023

Related U.S. Application Data
(60) Provisional application No. 63/156,028, filed on Mar. 3, 2021.

(51) Int. Cl. A61M 37/00

(52) U.S. Cl. CPC A61M 37/0015; A61M 2037/0023; A61M 2037/0046; A61M 2037/0053; A61M 2037/0061; A61M 2210/0612

(57) ABSTRACT

Microneedle arrays provided herein enable a minimally invasive platform for ocular drug delivery. Such a design and manufacture technique includes a conformable preformed interlocking mechanism configured about an elongated structure wherein the conformable preformed interlocking mechanism is arranged with a width that decreases in the needle tip direction and decreases in the base direction; and wherein each of the microneedles to include the conformable preformed interlocking mechanism is arranged to increase in area upon fluid tissue contact and decrease in area upon illumination with light.

### CROSS-REFERENCE TO RELATED APPLICATION

The present application claims under 35 U.S.C. § 119, the priority benefit of U.S. Provisional Application No. 63/156,028, filed Mar. 3, 2021, entitled “Microneedle array with a controllable locking feature,” which is incorporated herein by reference in its entirety.

### GOVERNMENT INTERESTS

This invention was made with government support under grant no. W81XWH-18-1-0137 awarded by U.S. Army Medical Research and Materiel Command (ARMY/MRMC). The government has certain rights in the invention.

### FIELD OF INVENTION

The embodiment herein relates to a photo-responsive microneedle configured with a preformed interlocking feature that can self-adhere to the tissue upon swelling and can be easily extracted upon deswelling after illuminated with light.

### BACKGROUND OF THE INVENTION

Age-related macular degeneration (AMD) and diabetic retinopathy (DR) are the leading causes of blindness in the US. Both AMD and DR are caused in part by over production of vascular endothelial growth factor (VEGF). In the presence of excessive VEGF, the capillaries begin to leak causing large molecules to form exudates to escape into the retina. This leakage causes edema in the surrounding tissues or abnormal growth of vessels. Clinical trials using VEGF inhibitors have shown superior results compared to laser photocoagulation for retinal and macular diseases.

Anti-VEGF therapies have been the standard treatment method for AMD and diabetic retinopathy (DR). Delivery of anti-VEGF agents needs to be localized to reduce systemic adverse effect and, therefore, is generally administered via intravitreal injections to bypass barrier properties of ocular tissues. The intravitreal elimination half-life of anti-VEGF drugs, such as Ranibizumab and Bevacizumab, is around 4-6 days. Thus, to maintain sufficient VEGF binding activities, regular intravitreal injections are required. Intravitreal injection is an invasive process. Most clinical trials use a treatment regime with regular monthly injections over the course of 2 years.

However, intravitreal injections have been associated with complications including cataracts, endophthalmitis, intraocular inflammation, retinal detachment, intraocular pressure elevation, and intraocular hemorrhage. Thus, intravitreal injection should be used as infrequently as possible. Other approaches of drug delivery to the eye, especially to the vitreous cavity present challenges. Topical administration, such as eye drops, cannot effectively deliver drugs to the vitreous cavity due to the physical barrier by the corneal epithelium and dynamic barrier by tear drainage.

Microneedles (MNs) offer a platform for minimally invasive drug delivery and are being investigated as an alternative to intravitreal injections. Microneedle arrays are medical devices used to deliver drugs to the human body via bypassing the skin barrier, namely the stratum corneum. Dimensions of MNs can range from 50 to 300 μm in diameter and 50 to 900 μm in needle height. Given MNs' small size, they provide a less painful and minimally invasive approach to delivering via the skin. Many clinical trials have been conducted to study the use of MNs in the delivery of macromolecules and vaccines with significant promise to achieve minimally invasive controlled drug delivery. Accordingly, it would be beneficial to employ MNs to deliver drugs into the eye for their sustained drug delivery capability. There are nearly 35 commercial devices employing the use of MNs.

One of the promising aspects of MNs is the sustained and controlled drug delivery capability. The number of microneedles in a microneedle array contribute to the capability of sustained and controlled drug delivery. Background information on drug delivery capability based on number of microneedles in the microneedle array, is described in “Hydrogel-Forming Microneedle Arrays for Sustained and Controlled Ocular Drug Delivery,” published in the Journal of engineering and science in medical diagnostics and therapy (Volume 3, 13 Oct. 2020) including the following: “Microneedles (MNs) provide a minimally invasive alternative to intravitreal injections and a promising means to sustainable ocular drug delivery. To optimize the sustained drug release profile and to ease the administration of the MN array to the eye, the number of MNs in an MN array and their layout need to be carefully selected. In this study, the drug release kinetics of MN arrays with varying numbers of MNs (8, 12, and 16) is studied over a four-week period. The MN arrays show a much more uniform drug release profile than the single injections. Only the 16-needle MN array fully released all the amount of loaded drug at the end of the 4-week period. Both 8- and 12-needle arrays showed a steady release rate over the 4-week period, which is the longest sustained release duration that has been reported . . . ”

As known in the art, hydrogels, because of the diverse and great polymer swelling characteristics of such materials have been used with great success as the fabrication material of MNs to achieve sustained drug delivery due to their ability to slow the diffusion of drugs into target systems. Moreover, sustained drug release with a zero-order release profile for four and up to six weeks can be achieved using such MN configurations.

Background information on particular aspects of hydrogels is described in “Mechanical properties of PNIPAM based hydrogels: A review,” published in the Journal Materials Science and Engineering: C (Volume 70, Part 1, Pages 842-855, January 2017) including the following, “ . . . Hydrogels formed by smart polymers have various applications. Among the smart polymers, thermoresponsive polymer poly(N-isopropylacrylamide) (PNIPAM) is very important because of its well-defined structure and property specially its temperature response is closed to human body and can be finetuned as well. Mechanical properties are critical for the performance of stimuli responsive hydrogels in diverse applications . . . ”

Background information on a swellable microneedle structure, is described and claimed in U.S. Pat. No. 9,549,746B2 entitled “Delivery device and method,” filed Sep. 29, 2008, to Woolfson et al, including the following, “A microprotrusion array for use in transport of a material across a biological barrier, wherein said array comprises a plurality of microprotusions composed of a swellable polymer composition.”

Background information on light-responsive hydrogels is described in “Molecular Design of Light-Responsive Hydrogels, For in Situ Generation of Fast and Reversible Valves for Microfluidic Applications,” published in the Journal Chemistry of Materials (2015, 27(17), 5925-5931) including the following. “ . . . Self-protonating gel formulations were exploited, wherein acrylic acid was copolymerized in the hydrogel network as an internal proton donor, to achieve a swollen state of the hydrogel in water at neutral pH. Light-responsive properties were endowed upon the hydrogels by copolymerization of spiropyran chromophores, using electron withdrawing and donating groups to tune the gel-swelling and shrinkage behavior. In all cases, the shrinkage was determined by the water diffusion rate, while for the swelling the isomerization kinetics is the rate-determining step. For one hydrogel, reversible and reproducible volume changes were observed. Finally, gel-valves integrated within microfluidic channels were fabricated, allowing reversible and repeatable operation, with opening and closing of the valve in minutes.”

Background information on a light-controlled hydrogel microneedle array, is described and claimed in China Patent No. CN112826791A entitled: “Light-controlled hydrogel microneedle array patch and preparation method thereof,” filed Jan. 13, 2021, to Sun et al, including the following, “ . . . The drug-containing micro-needle array is characterized in that a hydrogel matrix is used for loading drugs, the cyclodextrin and the azobenzene groups have high affinity under the visible light condition, a host-guest inclusion compound is formed for drug loading, the affinity of the cyclodextrin and the azobenzene groups is reduced under the ultraviolet light condition, the host-guest inclusion compound is opened, and the drugs are released. The two processes are reversible, so that a great number of medicines are released when the microneedle array is irradiated by ultraviolet light; when the visible light irradiates, the release amount of the medicine is greatly reduced, thereby realizing the controlled release of the medicine.”

One challenge for using MNs specifically for ocular drug delivery is the fixation aspect because of the sensitivity of the human eye and the limited area available for transscleral drug delivery. In transdermal applications of MNs, an adhesive backing is often used. However, chemical adhesives present inflammatory side effects to the eye. Hydrogel adhesives use covalent bonds to surface biomolecules but only provide limited adhesion. Sutures and staples offer firm adhesion to the eye but can lead to scarring and tissue damage and increase procedure time. Different challenges to securing the MNs can occur depending on which target location they are applied. For example, secretions such as sweat or saliva, and many fluids inside the body can interfere with the function of adhesives, thus making sustained and controlled drug delivery difficult.

To avoid the aforementioned side effects of common adhesion methods in MNs, researchers have looked to nature in their search for more suitable and well-defined geometrical microstructures that will provide higher adhesion forces and lower penetration forces. Many insects use needle-like organs to defend themselves against predators or threats in nature, and extract food or lay eggs in potential hosts. Some exhibit easy retrieval insertion tubes with minimum adhesion to very high adhesion force to prevent removal.

A bio-inspired microneedle design with an interlocking or barbed feature is an area of research that sought to address the issue of adhesion. A review study on bioinspired MNs concluded that many of the prior studies have focused on either the design of the microstructures or the use of vibrations, or movements, during insertion to achieve lower penetration forces. One of the most popular bioinspired designs of MNs was inspired by the mosquito's proboscis. These studies showed that barbed MNs have lower penetration forces than non-barbed MNs. There are also studies using MNs inspired by the North American porcupine quill and caterpillar spine to reduce insertion force. The effect of barbed features, working as mechanical interlocks, on self-adhesion of MNs has also been studied and shown some promising results. However, these added mechanical interlocking features significantly complicate the manufacturing process of MNs.

With respect to manufacturing, direct micromachining and micro-molding are the two most common approaches to fabricate MNs. MNs can also be fabricated using stereolithography-based or drawing processes. The direct micromachining approach, such as laser cutting and micro-milling, is used more frequently in the fabrication of metal MNs rather than polymer MNs due to the high cost of creating complex structures and these processes are not adequate for mass production. The micro-molding approach is usually used to fabricate polymer MN. Micro-molding has the benefit of having potential for mass production yet, may be challenged by the need for multiple steps and master molds to be fabricated from which the MNs will have to be separated. Adding barbs or interlocking features, as briefly discussed above, are at such a small scale that manufacturing them would require complicated techniques. For example, the barbs prevent the removal of the needles from the mold, causing demolding issues and requiring alternative mold solutions. Moreover, molds made by a rigid material will simply trap the barbed MNs in the mold. Some researchers skilled in the art attempted to use different approaches to fabricate barbed polymeric MNs. For example, the use of mold made of two pieces that allow separation of MNs that contain multiple layers of barbs from the mold has many steps and the final MN design is composed of one row of needles instead of an array. In addition, the multi-barb design is not suitable for ocular adhesion because as the needle penetrates the sclera, the inside is filled with vitreous humor that offers no anchor to the second barb, rendering it useless.

MNs with preformed interlocking features do allow for an increase in adhesion strength without increasing the required penetration force. Yet, MNs with preformed interlocking features present another challenge, i.e., the removal of the MN. While the bio-inspired interlocking features increase the adhesion force, they are not designed to be easily removed without causing pain or tissue damages. A high extraction force may cause retinal tear or detachment as well as an increased intra-ocular pressure which can lead to damages to the optic nerve. Thus, there is no existing MN design with self-adhesive preformed interlocking features for sustained drug delivery that can detach the MN array without scaring the target location including anywhere within the skin on the body, into the eye or any cell nucleus.

Accordingly, a need exists for a new microneedle (MN) design and fabrication technique that enables among other beneficial aspects, high resolution, ease of fabrication, cost effectiveness device with the potential for mass production. The embodiments disclosed herein addresses such a need by the use of a photo-responsive hydrogel MN design with a self-adhesive preformed interlocking feature which swells on insertion and in which the extraction can be controlled through the stimulation of light for sustained drug delivery to a target location and a new fabrication technique for such MN design.

### BRIEF SUMMARY OF THE INVENTION

An aspect of the present invention is directed to a microneedle array, that includes: a plurality of microneedles, wherein each of a respective microneedle of the plurality of microneedles further comprises: a base; a needle tip; an elongated structure therebetween the base and the needle tip; and a preformed interlocking feature configured about the elongated structure wherein the preformed interlocking feature is arranged with a width that decreases in the needle tip direction and decreases in the base direction.

A second aspect of the embodiments herein is directed to a method of drug release, including: inserting at least one or more microneedles each into a target location wherein each of the one or more microneedles has a configured preformed interlocking feature; increasing the width of the preformed interlocking feature upon a fluid tissue contact so as to secure each of the one or more microneedles at the target location; releasing a self-contained drug disposed in each of the one or more microneedles; and illuminating the one or more microneedles using a light source, wherein the width of the preformed interlocking feature decreases so as to enable the extraction of the one or more microneedles from the target location.

Another aspect of the embodiments is directed to: microneedle array fabrication process, comprising: molding a microneedle array, wherein a respective microneedle of the microneedle array has a configured preformed interlocking feature; casting a fabrication mold of the microneedle array; disposing a hydrogel concentration into the fabrication mold; centrifuging the fabrication mold; and subjecting the fabrication mold having the disposed hydrogel concentration with one or more freeze-thaw cycles.

Beneficial aspects of the photo-responsive hydrogel microneedle arrays disclosed herein include the capability of self-adhering to the application site upon swelling and can deswell for easy removal when illuminated with light. Beneficial experimental results show a significant decrease in extraction force after the microneedle of 20% spiropyran-conjugated NIPPAM is illuminated with light for 15 minutes. At the same time, the width of the preformed interlocking feature also deswelled by 20% due to the photo-responsive behavior.

As stated herein before, such arrangements and fabrication of such arrangements beneficially allow for longer sustained drug delivery and easy removal, without the need of surgical removal, when drug delivery is completed and enables use in different target locations in the human body where chemical adhesives use will not be possible due to bodily fluids. Such a new concept opens the door to many possibilities of sustained drug delivery.

### BRIEF DESCRIPTION OF THE FIGURES

Many of the drawings submitted herein are better understood as provided by the original images, which are not best depicted in patent application publications at the time of filing. Applicant considers the recreated images, as shown by the drawings, as part of the original submission and reserves the right to present such images of the drawings in later proceedings.

FIG. 1 illustrates the concept of the preformed interlocking feature of the microneedle design herein.

FIG. 2 shows the penetration forces for microneedles with no feature, design 1, and design 2 tested on phantom, and design 1 tested on rabbit's eyes.

FIG. 3 shows the adhesion forces for microneedles with no feature, design 1, and design 3, and design 1 tested on rabbit's eyes.

FIG. 4 shows the penetration forces of microneedles with PVA concentrations of 10%, 12%, and 16% w/w.

FIG. 5A-5D shows the swelling of a microneedle at 0, 2, 6, and 10 min respectively.

FIG. 6 shows the percent increase of preformed interlocking feature width and area overtime due to microneedle swelling.

FIG. 7 shows the minimal degradation of microneedles after soaking in water for 4 weeks.

FIG. 8A shows an illustration of the microneedle geometry with preformed interlocking feature and its design parameters.

FIG. 8B also shows the illustration of the microneedle geometry of an exploded view of the preformed interlocking feature and its design parameters.

FIG. 9A shows step 1 for a microneedle fabrication process disclosed herein.

FIG. 9B shows step 2 for a microneedle fabrication process disclosed herein.

FIG. 9C shows step 3 for a microneedle fabrication process disclosed herein.

FIG. 10 shows an example graphical representation of an actual image of a fabricated microneedle array.

FIG. 11A shows an experimental setup for measuring penetration and adhesion forces.

FIG. 11B shows an exploded view of a portion of the experimental setup shown in FIG. 11A.

FIG. 12A shows an illustration of the application and extraction process herein of the self-adhesive microneedle with a photo-responsive feature for easy removal. FIG. 12A, in particular, shows insertion into an eye of a subject.

FIG. 12B further illustrates the application and extraction process of the self-adhesive microneedle with a photo-responsive feature for easy removal. FIG. 12B, in particular, shows response of the microneedles in the array swelling and self-adhering.

FIG. 12B′ further illustrates the application and extraction process of the self-adhesive microneedle with a photo-responsive feature for easy removal. FIG. 12B′, more specifically shows as insertion into an eye of a subject comes into play, the needles in the MN array, including the preformed interlocking feature, starts to swell and each of the microneedles in the array self-adheres to the eye.

FIG. 12C further illustrates the application and extraction process of the self-adhesive microneedle with a photo-responsive feature for easy removal. FIG. C, more specifically, illustrates deswelling of the array upon light illumination.

FIG. 12D illustrates the easy removal of the deswelled MN array from the eye.

FIG. 13 shows the force measurement versus insertion distance for a microneedle.

FIG. 14 shows an extraction force of microneedles study with 80% PVA-20% Spiropyran-conjugated poly (NIPPAM) gels (PNS) (w/w) and 90% PVA-10% PNS under light illumination and under no light illumination. * Indicates significant difference (p<0.05).

FIG. 15 shows the extraction force microneedles with 90% PVA-10 PNS (w/w) under light illumination and under no light illumination.

FIG. 16 shows the penetration forces for microneedles with PVA/PNS 20%, PVA/PNS 10% and PVA 100% under no light stimulation.

FIG. 17A shows microscope images of swelling and deswelling of a single microneedle. FIG. 17A, in particular, shows the MN in its initial state at time T=0 min.

FIG. 17B also shows microscope images of swelling and deswelling of a single microneedle. FIG. 17B, in particular, shows swelling of a MN at time T=4 min.

FIG. 17C also shows microscope images of swelling and deswelling of a single microneedle. FIG. 17C, in particular, shows swelling of a MN at time T=10 min in.

FIG. 17D also shows microscope images of swelling and deswelling of a single microneedle. FIG. 17D, in particular, shows that light illumination applied to the MN at the 10 minutes mark for 14 minutes. Surprisingly and unexpectedly, the MN observed at time T=24 min in FIG. 17D was observed to have a de-swollen geometry with decreased width and decreased area as compared to swollen width and swollen area of FIG. 17C.

FIG. 18 shows the swelling and deswelling kinetics of a single microneedle.

### DETAILED DESCRIPTION OF THE INVENTION

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

### General Description

To reiterate, the device(s) disclosed herein locks itself to the target location with no need for external adhesives, sutures or staples using a preformed interlocking feature at an arranged position (e.g., near or at the waist) of the MN and allows for easy extraction when the preformed interlocking structure deswells on being subjected to light. Such an arrangement allows for longer sustained drug delivery to a desired site of action within the eye without the need for surgical removal. The MN design, often an array of such MNs, as presented herein, enables use in different target locations in the human body including the eye and even within the skin or the cell nucleus where chemical adhesives use would not be possible due to bodily fluids.

### Specific Description

It is to be appreciated that the needles and thus arrays described herein are preferably configured from hydrogels, because as known in the art, they are safe for medical applications, biodegradable, and easily modified. Moreover, hydrogels can be easily synthesized by chemical reactions and by using different hydrogel concentrations. Hydrogels herein, are often synthesized using polymers such as, but not limited to, a natural polymer or a synthetic polymer. Natural polymer examples include but are not limited to proteins or polysaccharides. Synthetic polymers include, but are not limited to, polyvinyl alcohol (PVA), polyethylene glycol (PEG) or polyacrylic acid (PAA). However, for illustrative nonlimiting purposes, the present invention often, but not necessarily utilizes PVA herein in various concentrations to minimize insertion forces. Accordingly, because of the hydrophilic structure of the polymers configured for the embodiments herein, such materials take up fluids into their three-dimensional polymeric network and swell when inserted into desired tissue structures, such as the eye. due to the eventual presence of an enveloping fluid, i.e., vitreous humor.

Moreover, the hydrogels utilized herein are configured to be photo responsive, as better detailed infra. In particular, the microneedle arrays (MNs) as noted herein self-adhere to an application tissue site upon swelling and can deswell for easy removal when illuminated with light. The photoresponsive hydrogel microneedle arrays, as disclosed herein, are preferably configured from a mixture of polyvinyl alcohol and spiropyran-conjugated. N-isopropylacrylamide (NIPPAM). As an example embodiment, results show a significant decrease in extraction force after the microneedle of 20% spiropyran-conjugated NIPPAM was illuminated with light for 15 min. At the same time, the width of the preformed interlocking feature also deswelled by 20% due to the photoresponsive behavior.

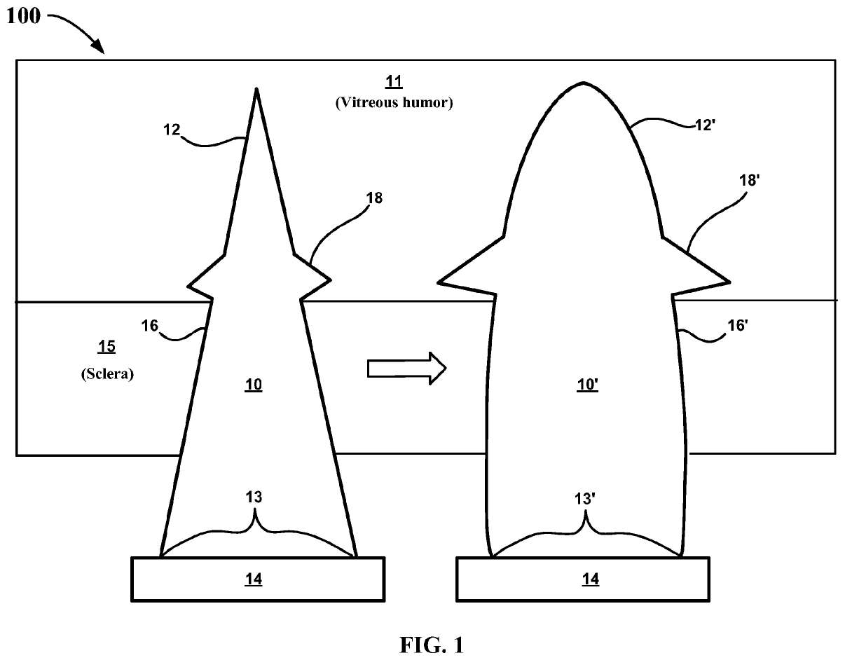

Turning specifically to the drawings, FIG. 1, as generally referenced by the numeral 100, generally illustrates the workings of the present invention with unprimed reference numerals indicating a structure prior to swelling and primed numerals indicated a MN after fluid contact and thus self-adhesively positioned into place by way of construction. Specifically, the graphical representations shown in FIG. 1 is that of a microneedle (MN) 10 prior to coming into contact with a fluid 11 (such as, for example, vitreous humor as denoted in FIG. 1) and that of a resultant swollen MN 10′ upon fluid 11 contact, as to be detailed hereinafter.

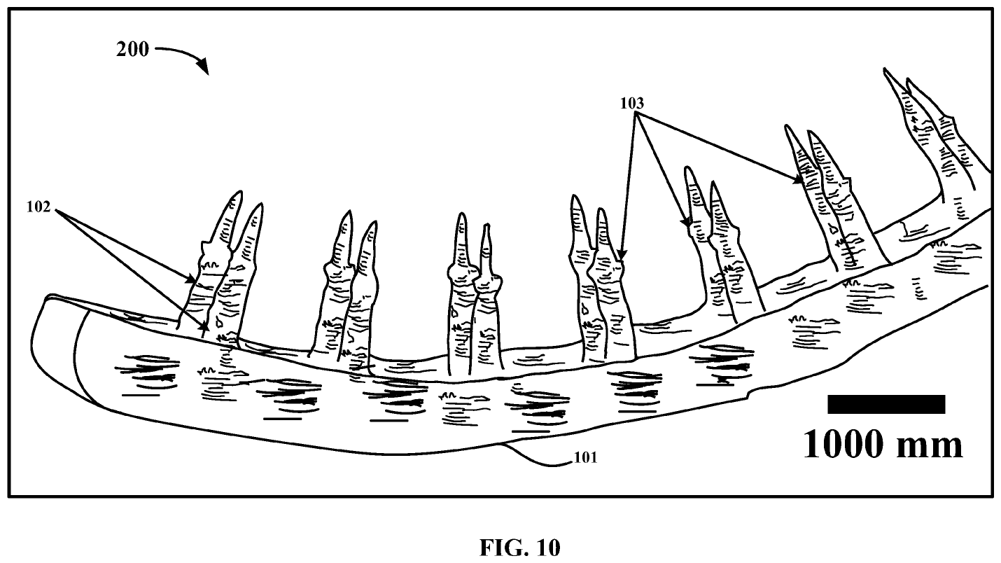

It is to be noted that the configured microneedles, as shown in FIG. 1, are arranged with a needle tip 12, 12′ having a perforation at the tip (not shown) for drug delivery and a body, (i.e., an elongated structure 16, 16′ therebetween the needle tip 12, 12′ and a resultant base 13, 13′) all of which is coupled to a substrate 14 (e.g., via a molding process). A novel aspect of the present invention is a preformed interlocking feature 18, 18′ that is integrally configured at a designed position anywhere along the elongated structure 16, 16′, which is often, but not necessarily, at approximately adjacent the waist of the MN 10. Such a MN is coupled to a substrate 14 and if configured along with other MNs, such an arrangement forms a microneedle array (e.g., see FIG. 10), as disclosed herein.

In an example method of operation, a particular MN 10 and often two or more MNs, often arranged as an array of microneedles, each having respective preformed interlocking features 18, is/are introduced in a controlled fashion into the ocular tissue of a subject (e.g., the sclera) with a depth of penetration determined primarily by the length of the MNs 10. Generally, as the microneedles 10 (e.g., arranged in an array) are introduced into the tissue, the depth of penetration is controlled by generally, for example, controlling the degree of tissue deformation of the eye.

Upon penetration of a desired tissue, e.g., the sclera, of each of the microneedles MNs 10′ to include preformed interlocking features 18′, and upon envelopment by bodily fluids, often the vitreous humor 11, physiochemical processes of the MNs 10′ via their hydrogel construction induces swelling, as illustrated in FIG. 1. Importantly, the preformed interlocking feature 18′, which is integrally configured with each respective MN 10′, also swells and the overall construction enables the array of MNs 10 to be removeably affixed in place by such a design. Specifically, as the preformed interlocking feature 18′ is induced to swell, it aids the overall respective microneedle to lock (i.e., self-adhere) onto tissue (the sclera 15) so as to secure the particular MN 10 in place.

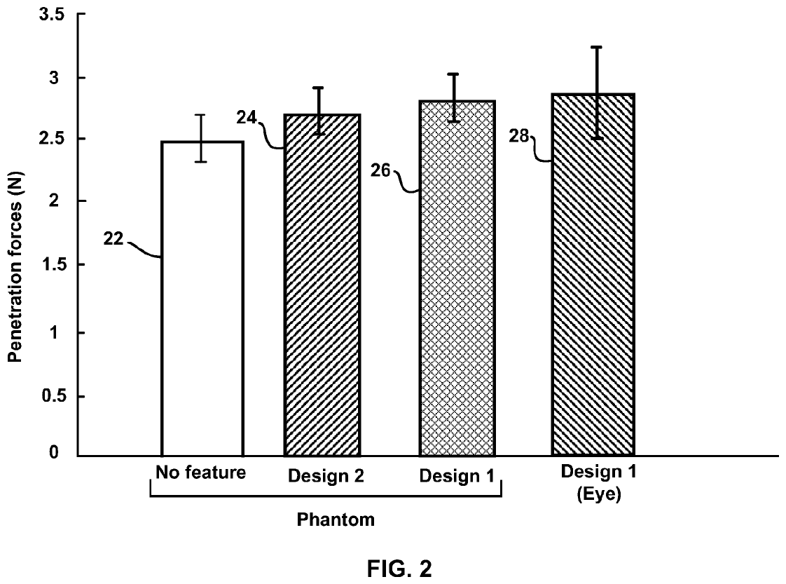

FIG. 2 illustrates average penetration forces data by MNs disclosed herein with no preformed interlocking feature 22, a (design 1 24) with a larger preformed interlocking feature width than that of design 2 26. Accordingly, design 1 24 and design 2 26 represent different widths of the preformed interlocking feature at approximately adjacent the waist of the MN. The far-right bar 28 shows the penetration force of design 1 tested on rabbits' eye tissues.

While there is no statistically significant difference among all MN designs, the data of design 1 26 indicates that a bigger preformed interlocking feature provides for a slightly higher penetration force than those of MNs with no preformed interlocking feature 22 and even to that of design 2 24 configured with a lesser initial preformed interlocking feature width. MNs with no feature show the lowest penetration force 22; however, the difference between design 1 26 and no feature 22 is less than 0.4 N. Comparable penetration forces were obtained by the sclera-mimicking phantom and the rabbits' eyes using design 1 28, demonstrating that the sclera-mimicking phantom can be used as more assessable material as an alternative to actual scleral tissues.

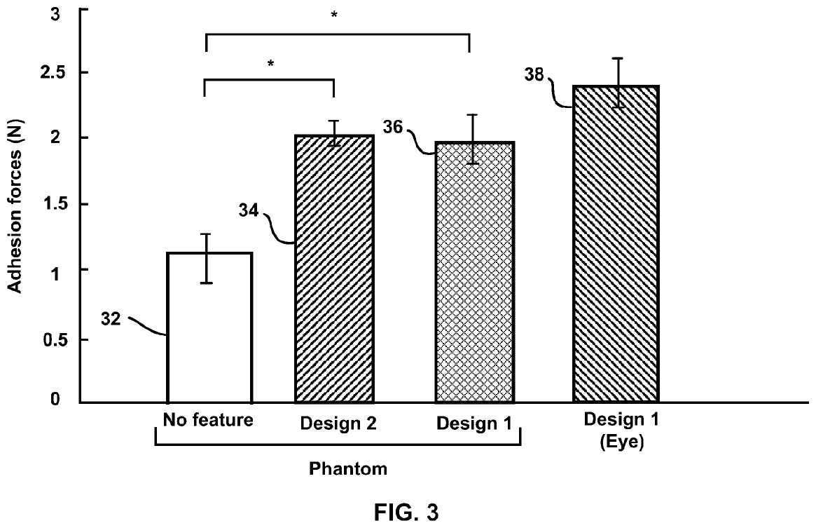

FIG. 3 shows resultant data for the average of 10 adhesion strength tests on different MN designs, similar to that shown and discussed in FIG. 2. The adhesion strength is quantified by an adhesion force defined as the maximum measured force while pulling the inserted MNs out of the testing material. The first three bars 32, 34 and 36 are the results tested on a sclera-mimicking phantom and the last bar 38 is the result tested on rabbits' eye tissues. The results show that designs 1 36 and 2 34 had an unexpected and surprisingly significantly higher, more than 80%, adhesion force than the MN with no feature 32. It is to be noted that there is no significant difference between design 1 tested on the sclera-mimicking phantom 36 and design 1 tested on ex vivo rabbit's eye tissues 38 and the resulting adhesion forces were comparable. The results also show that the design 1 tested on rabbit's eyes in vitro had higher adhesion forces. * indicates significant difference (p<0.05) between the adhesion forces tested on the different MNs. It is also to be noted that a MN array with a similar length and size tested on rabbits' eye tissues showed no intraoperative and postoperative complications, as well as no acute ocular inflammations. Therefore, an example MN array configured with a 1500 μm length does not cause significant damages to the eye.

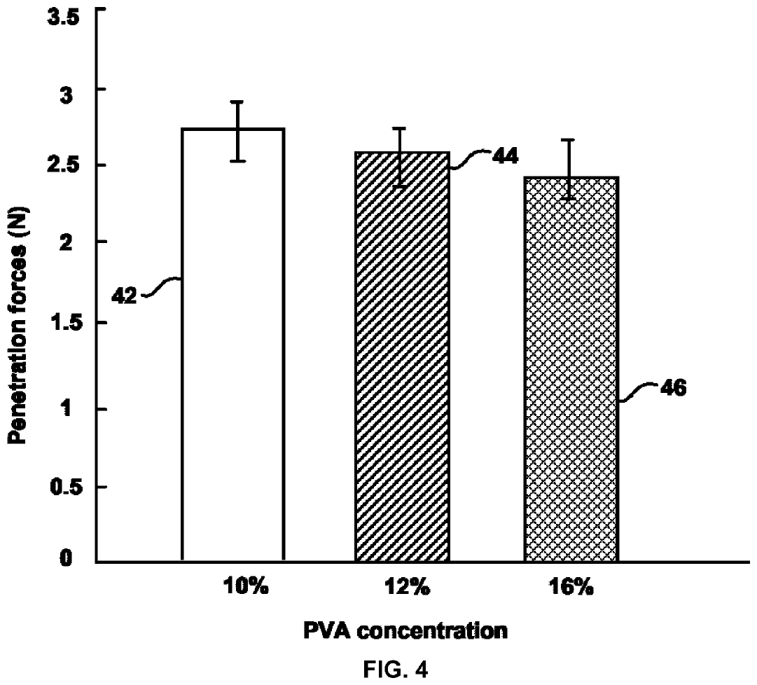

FIG. 4 illustrates penetration forces by MNs manufactured with different polyvinyl alcohol (PVA) concentrations. As shown in FIG. 4, the bars 42, 44, and 46 represent the penetration forces by MNs manufactured with 10% w/w, 12% w/w and 16% w/w PVA concentrations respectively. The penetration force decreases as the PVA concentration increases from left most bar to the right most bar. It is to be noted that a total of 16% w/w 46 shows a beneficial lowest penetration force and such a PVA 16% w/w concentration was utilized henceforth for working example purposes.

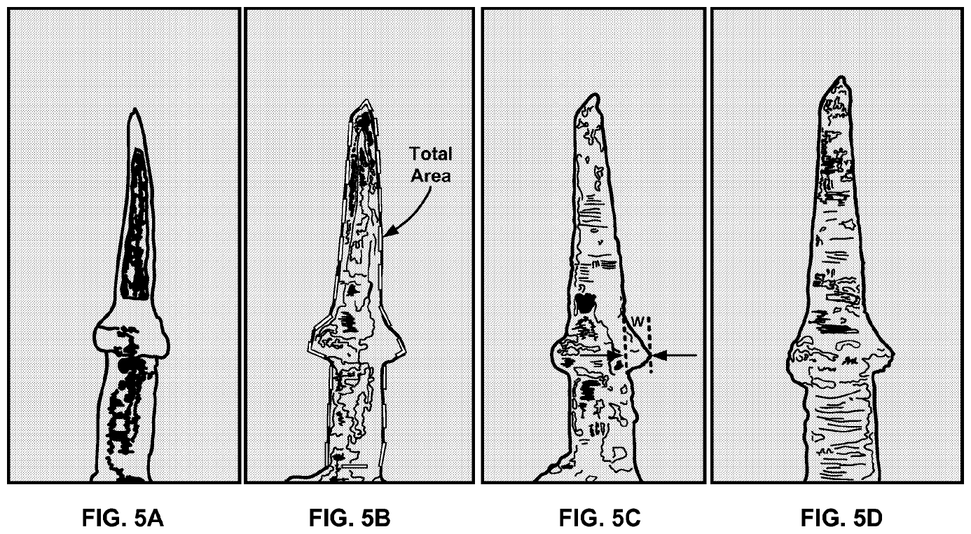

FIG. 5A through FIG. 5D illustrates graphical representations from real images of the progression of swelling of MNs over time while being immersed in a vitreous humor-mimicking gel. FIG. 5A shows the MN at time T=0 min. The MNs begin swelling and the total area (e.g., dashed whited outline shown and denoted in FIG. 5B) and preformed interlocking feature width (see dotted lines in FIG. 5C and as denoted by the letter w) of the MN increases with time as observed at time T=2 min in FIG. 5B, at time T=6 min in FIG. 5C and at time T=10 min in FIG. 5D. These measurements were further analyzed to quantify the swelling kinetics of MNs.

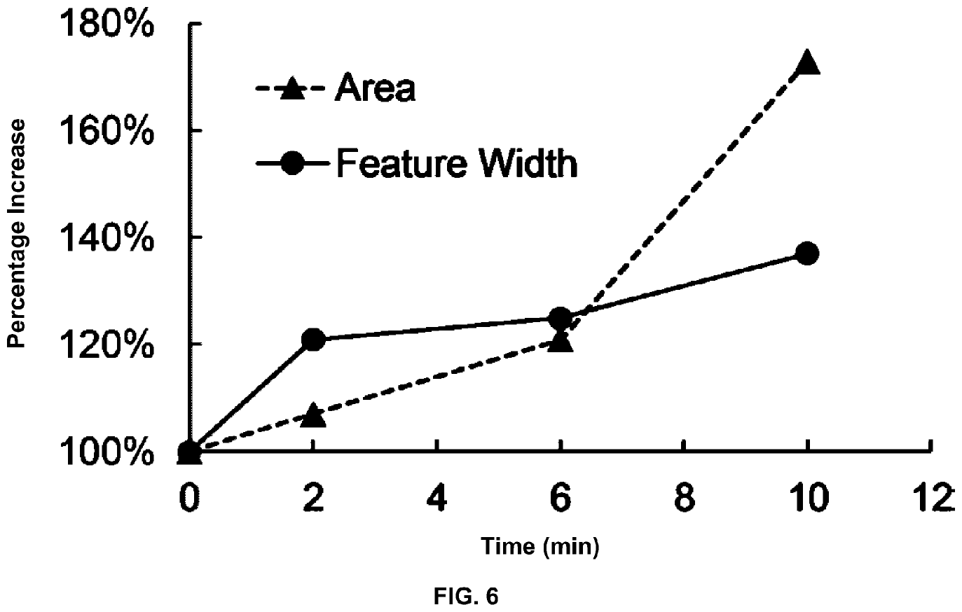

FIG. 6 shows the increase of the total area and feature width of an example MN over time. As can be seen, as discussed above in FIG. 5B and as illustrated by the data in FIG. 6, the feature width (represented by circles) increases rapidly by about 20% in 2 min. The increase slows down and increases by about another 10% till 10 min, which is represented by the graphical representation of FIG. 5D. Turning back to FIG. 6, the total area (represented by triangles) slowly increases by 20% over the first 6 min (also refer to FIG. 5C) and then further increases by another 55% up until 10 min (also refer to FIG. 5D).

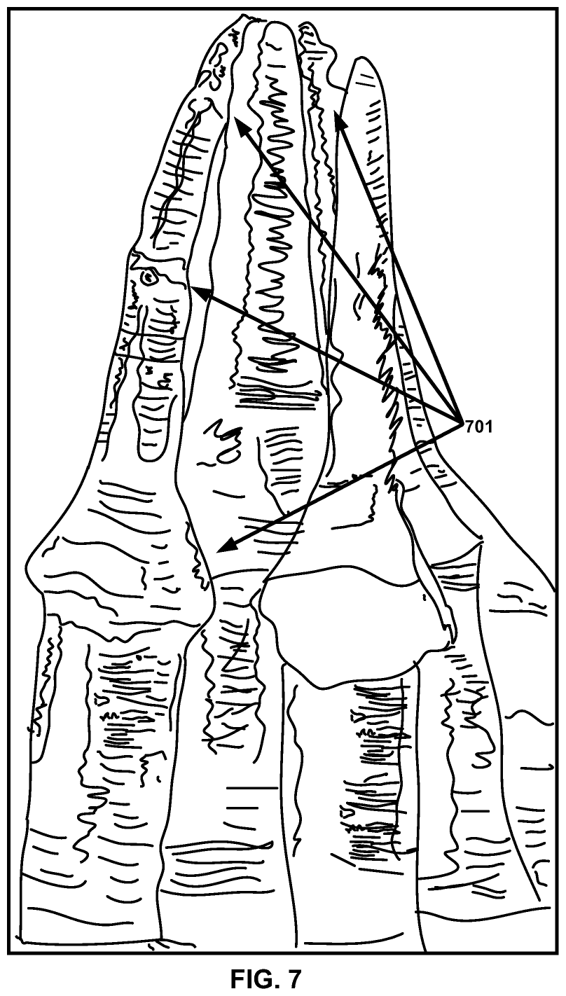

The MN was left immersed in the vitreous-mimicking gel for a period of 4 week and observed. FIG. 7 is a recreated graphical representation of the real image for MNs 701 after the 4-week degradation period. Only minor signs of degradation were observed in the image (and as best recreated graphically), but the needles remained intact. This beneficially demonstrated that the PVA material used herein to fabricate the MN can be used for sustained drug delivery, without losing the structure integrity due to dissolving.

In the first example embodiment presented herein, the MN design with a novel preformed interlocking feature achieves self-adhesion as presented. MNs with this preformed interlocking feature results in an 80% increase in adhesion strength compared to MNs with no feature. Incorporating the preformed interlocking feature only slightly increases the required penetration force. It was found that experiments done using the sclera-mimicking phantom and ex vivo scleral tissues have similar results on adhesion force and penetration force. Thus, the sclera-mimicking phantom is used as an effective alternative for scleral tissues that are not easily assessable. In addition, the MN fabrication process presented herein employs the use of 3D printing that allows for rapid design changes. The fabrication process involves a spin casting process which is an easy and robust process to produce the final MNs and has the potential to be scaled up for industrial and clinical applications.

### Experimental Section

A. Design of MN with Preformed Interlocking Features

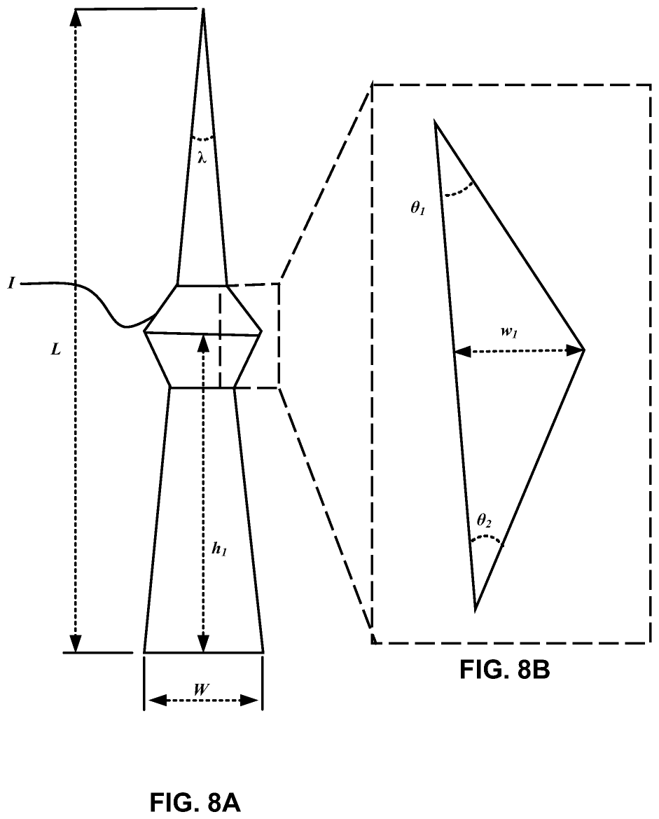

FIG. 8A and FIG. 8B (an exploded view of the preformed interlocking feature) illustrate the geometry of the MNs herein with the preformed interlocking feature (denoted by the Letter I in FIG. 8A). Table 1 below includes design parameters for the two example designs presented herein as designs 1 and 2, as were discussed above. For both designs, the total height (denoted as L in FIG. 8A) of the MN is an example 1500 μm in effective length and the base (denoted as W in FIG. 8A) is an example 275 μm in diameter. While effective lengths and base diameters are discussed to demonstrate working embodiments, it is for illustration purposes, and it is be noted that other lengths from about 500 μm up to 2000 μm in effective lengths and with base diameters of 200 μm up to 400 μm and different widths w₁ ranging from 40 μm to 80 μm can also be utilized herein.

Microneedles (MN) disclosed herein are configured with a needle tip with an interior angle of (denoted as λ in FIG. 8A). The interlocking preformed feature I on the MN is often configured at approximately adjacent the waist of the MN at a height (denoted as h₁ in FIG. 8A) from the base of diameter W. Herein, designs 1 and 2 (as were discussed above) have a different width (denoted as w₁ in FIG. 8B) of the preformed interlocking feature I. As were seen for both designs 1 and 2, as were discussed above, the exist angles (denoted as θ₁ and θ₂ in FIG. 8B) on the preformed interlocking feature I has a smooth angle to prevent damages to the surrounding tissues during extraction, without compromising the adhesion strength. Having a bigger exist angle can increase the adhesion strength, but also increase the difficulty for extraction and vice versa. While the presented designs have exist angles of 23° and 30° and an interior angle of 10.48°, it is to be noted they can be any angle that comports with the design parameters of the present invention such as, for example 15° up to 40° for the exist angles and any angle for the interior angle that works with the embodiments herein so as to comport with the scope and spirit of the present invention.

TABLE 1
Design parameters of MNs with preformed interlocking feature.

| Parameter | Design 1 | Design 2 |
|---|---|---|
| θ₁ | 30° | 23° |
| θ₂ | 30° | 23° |

| | |
|---|---|
| λ | 10.48° |
| L | 1500 μm |
| W | 275 μm |

| | | |
|---|---|---|
| w₁ | 69 μm | 51 μm |

| | |
|---|---|
| h₁ | 750 μm |

### B. Fabrication of the MN Patches

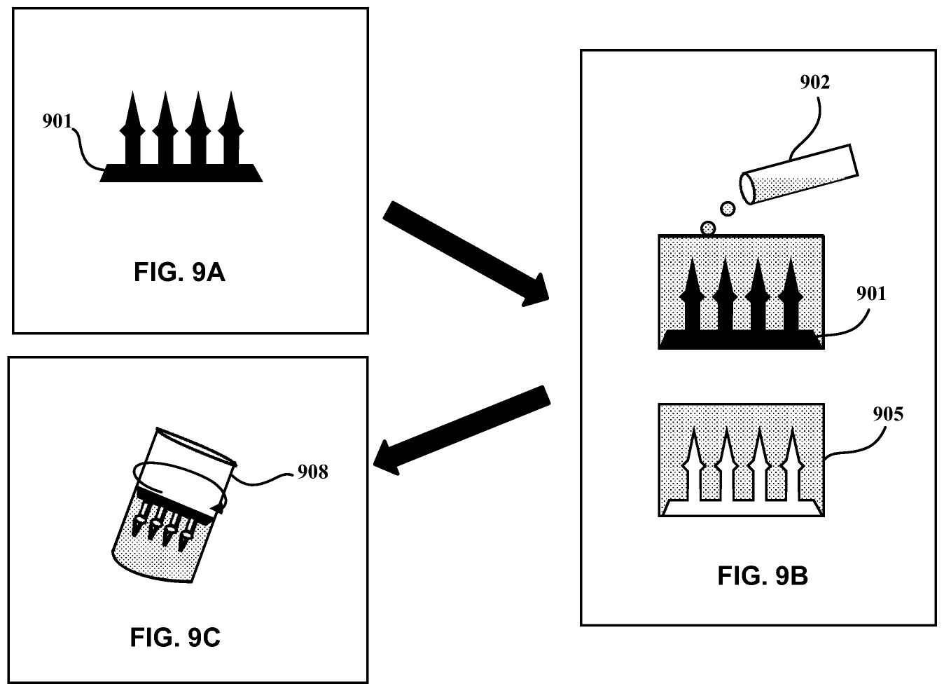

Turning now to the flowchart of the fabrication procedure shown in FIGs. FIG. 9A-9C. The fabrication procedure herein includes 3 major steps, as shown FIG. 9A, FIG. 9B, and in FIG. 9A. In step 1 (FIG. 9A), computer-aided design models of the MN array were created and converted into input files for a digital light processing-based 3D printer (B9 Core 530, B9Creations, Rapid City, SD, USA) followed by printing a master mold 901. The master mold of the MN array includes a group of MNs, wherein each respective MN has the preformed interlocking feature. SYLGARD 184 (Dow Corning) is used to cast soft fabrication molds, following an informed procedure known to those skilled in the art. The elastomer used herein for casting is made of two parts: one is a curable silicone, and the other is a curing agent. The ratio used is 10:1 elastomer to curing agent, respectively. The elastomer is first mixed in a centrifuge unit (NuWind NU-C200R, NuAire, Plymouth, MN, USA) at 2500 rpm for 4 min and degassed for 10 min to create a mixture. In step 2 (FIG. 9B), a mixture 902 is poured about the 3D-printed master mold 901 to create a fabrication mold 905.

A beneficial example polymer PVA (Mw=146 000-186 000, 99% hydrolysis; Sigma-Aldrich, St. Louis, MO, USA) was mixed with deionized water to make aqueous blends of PVA at concentrations of 10%, 12%, and 16% w/w and are stirred and heated under nearly 90° C. until the solutions became clear. The PVA solutions were then poured into the fabrication mold 905. The fabrication mold 905 was then vacuumed and centrifuged using a centrifuge unit 908 in step 3 (FIG. 9C). They were then subjected to seven freeze/thaw cycles (freeze for 8 h at −20° and thaw for 5 h at 25° C.) to make the hydrogel to be utilized herein. The hydrogel MNs are thereafter removed from the fabrication molds. The hydrogel MNs are thereafter removed from the fabrication molds. It is to be noted that as an aspect of the embodiments presented herein, the preformed interlocking feature of each of the plurality of microneedles in a microneedle array is constructed from a photo-responsive compound coupled to a hydrogel, and wherein the remainder of each of the plurality of microneedles are configured from a non-photo-responsive hydrogel.

FIG. 10 shows a graphical representation of the real image for a fabricated MN array patch (note the recreated 1000 mm scale). As illustrated and as part of the process discussed above in FIGS. 9A-9C, a resultant fabricated MN array, as generally referenced by the numeral 200, has a substrate 101 with a curvature that matches the eye curvature to ease adhesion. The fabricated MN array for the embodiment presented herein thus entails a substrate 101 with two rows or more of needles (a needle of row 1 and a needle of row 2 are represented by the reference numeral 102) placed on the substrate 101 and each respective row can contain eight or more needles 103 (for simplicity, 103 points to only 3 of such represented 8 needles). Example dimensions shown in FIG. 10 of a MN patch 200 for the embodiment presented herein are 7.6 mm in length, 1.71 mm in width, and 2.32 mm in height on the curved base but are for illustrative purposes of a working embodiment only and can vary in length, width, and height depending on varying design parameters.

### C. Preparation of Sclera-Mimicking Phantom and Vitreous Humor-Mimicking Gel

The sclera-mimicking phantom was prepared following a standard procedure known to those skilled in the art. In short, polycaprolactone is dissolved in chloroform with 5% w/v. It is then spun in a centrifuge to achieve a homogeneous mixture. To prepare a vitreous humor mimicking gel, PVA was mixed with a solution containing 2.5% w/v gelatin and water. The PVA solution to the gelatin water mix is of the percentage of 31.5% w/v. The solution was stirred at 70° C. A total of 50 μL of 1 M HCl was then added and the mixture was stirred for 30 min at 50° C. The gel was then stored in a sealed container and left to cool to room temperature.

### D. Experimental Setup

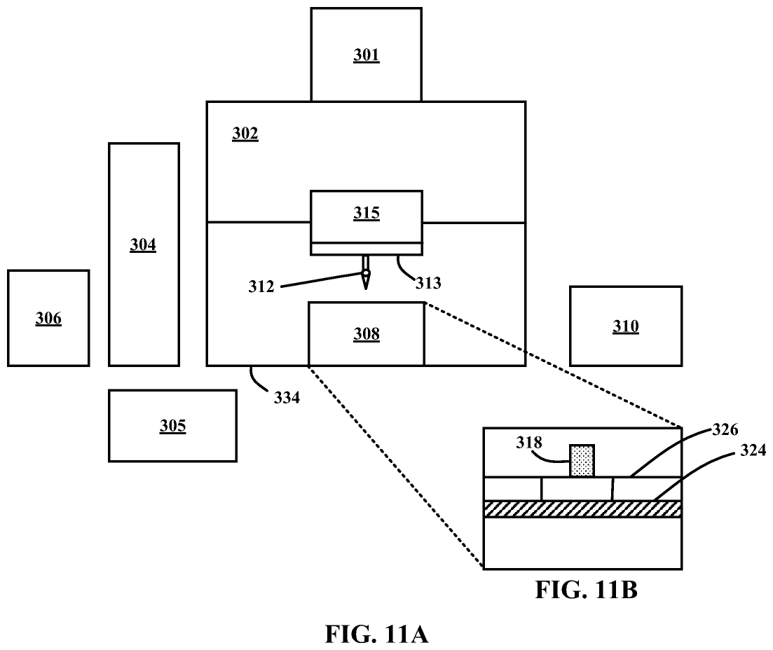

FIG. 11A and FIG. 11B show a graphical representation of the experimental setup and exploded portioned view of the setup for the measurement of both penetration and adhesion forces so as to provide data for the disclosure herein. A 3D printer (LulzBot TAZ 6, Aleph Objects, Loveland, CO, USA) was converted to a 3-axis linear stage motion system (302, 334). The MN patch 312 (it is to be noted that for experiment purpose, only 1 microneedle is shown herein) was attached to a MN holder 313 that is connected to the printhead carriage 315 of a 3D printer 302. The sclera-mimicking phantom or an ex vivo eye 318 (as shown in the exploded view of FIG. 11B) is fixed by an acrylic mount on top of a load cell 324 (Miniature Low Profile Tension Link Load Cells model #LC703, OMEGA, Norwalk, CT, USA) that is used to measure the penetration or adhesion force. A block 308 includes the sclera-mimicking phantom or an ex vivo eye 318, and an eye holder 326 on the load cell 324. A linear stage 334 is used to drive the MN patch 312 into the sclera-mimicking phantom or the eye 318 at a speed of 100 mm min⁻¹. Intact eyes used in this experiment were extracted surgically from recently euthanized New Zealand rabbits averaging 5 kg in weight and kept frozen at −20° C. before use. The eyes were thawed for 2 h and were blotted dry before conducting the experiment. Handling of the ex-vivo tissue samples has been in accordance with the IACUC approved protocol (ASAF #6465). A LabVIEW program is used to record the measurements and control the linear stages. The data sampling rate is 315 Hz. Additional components in the experimental setup include a computer 301 and its CPU 304, a power supply unit 306, and a data acquisition system 305 (Model #USB-1608G, Measurement Computing, Norton, MA, USA).

### E. Measurement of Penetration Force

First, penetration forces of MNs made by 10%, 12%, and 16% w/v PVA were tested to determine the optimal concentration of PVA with the minimal penetration force. Penetration forces of MNs with no feature, design 1, and design 2 were tested on sclera-mimicking phantom. Lastly, MNs with design 1 were tested on full intact rabbit's eye tissues.

### F. Measurement of Adhesion Force

The MN patches 312 were glued to the MN holder 313 using a cyanoacrylate glue. The MN patches 312 were pushed into the samples and held for 10 min to allow for swelling. The swelling was made possible by filling the holding box with vitreous humor-mimicking gel and placing, and holding, the mimicking phantom on top. As the patches 312 penetrated the mimicking phantom 318 layer the tips then have access to the humor mimicking gel. After 10 min, the MN patches are pulled in the opposite direction at a rate of 100 mm min⁻¹. MNs with no feature, design 1, and design 2, as discussed above, were tested on the sclera-mimicking phantom. Also, design 1 MNs were tested on fully intact rabbits' eyes with its muscular connections.

### G. Characterization of MN Swelling Kinetics

An acrylic box is made and filled with the vitreous humor-mimicking gel and a 1 mm thick sclera-mimicking phantom is fastened to seal the box from the top side. One row of the MNs is cut and glued to a metal plate. Using the linear stage, the row of MN is inserted through the phantom layer, coming in contact with the vitreous humor-mimicking gel. MNs absorb water from the gel and swell. The shape change is recorded using an inverted microscope at times of 0, 2, 6, and 10 min as shown in FIG. 5. ImageJ is used to analyze the images to quantify the swelling kinetics. In addition, a degradation test is performed to evaluate the sustainability of the MN material. The MNs are submerged in water for 4 weeks. After that, the geometry of MN is examined under microscope.

### H. Statistical Analysis

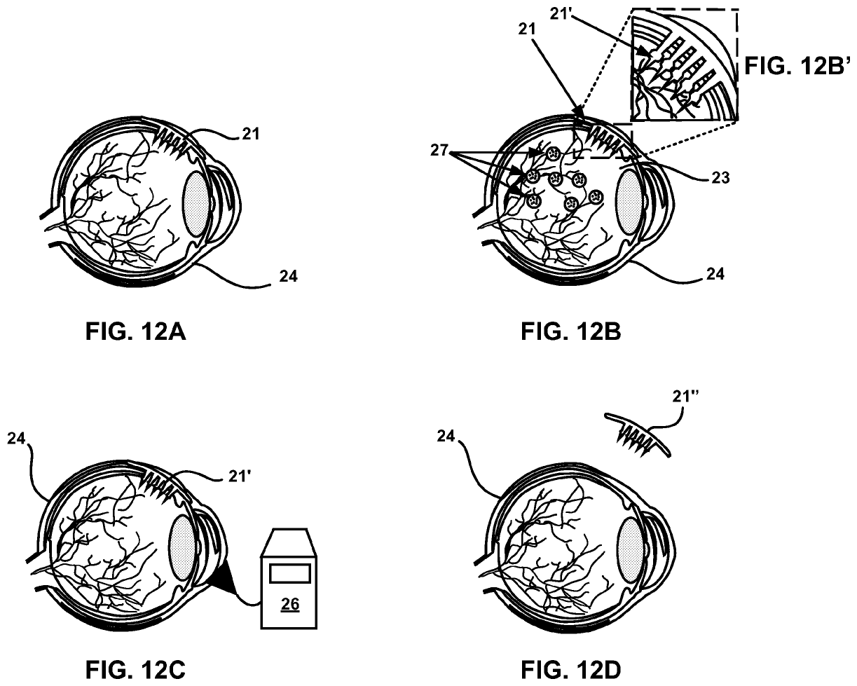

ANOVA tests are performed using Excel to assess the statistical significance of the differences between groups of no feature, design 1, and design 2. The second example embodiment herein thus entailed a strategy for easy removal of the self-adhesive hydrogel microneedles (MNs). Particularly, the use of a photo-responsive hydrogel microneedle (MN) with swelling capability and deswelling controllable through the stimulation of light. FIG. 12A through FIG. 12D illustrates an overview of the process of swelling and deswelling of the photo-responsive hydrogel MN. In FIG. 12A, the MN array 21 is inserted into a subject eye 24, i.e., the sclera and often perpendicular to the sclera. As can be seen in FIG. 12B, more specifically, as shown by the exploded view of FIG. 12B′, as the preformed interlocking feature 21′ at approximately adjacent the waist of each respective MN of the array 21 comes in contact with the vitreous humor 23 (see FIG. 12B), the entire needles in the MN array 21, including the preformed interlocking feature 21′, starts to swell and each of the microneedles in the array 21 self-adheres to the eye.

As a pre-requisite step, the drug of choice to be delivered into the eye is initially dissolved in water to create a drug solution with the intended drug concentration. Drug(s) is/are disposed in each MN in the MN array 21 via absorption of the drug solution. The drug(s) disposed in the microneedles of the array 21 infuses through the sclera and thereafter adjacent tissues, as known in the art, in a controlled manner, often in a time frame designed to minimize patient discomfort. Moreover, it is to be appreciated that drugs delivered by the embodiments herein can also be administered to the other ocular regions, such as, for example, the macula. In addition, a number of eye diseases and disorders can be treated using the embodiments described herein, such as, but not limited to, glaucoma, diabetic macular edema, uveitis, macular degeneration, and genetic diseases. In one embodiment, the delivery methods and and such embodiments herein can also be used for gene therapy applications

Turning back to FIG. 12B, the MN array 21 thus is configured to release a drug 27 (3 labelled for simplicity) in a controlled manner sustained manner into the eye 24. The release of the drug 27 from a microneedle configured in an array 21 involves the absorption of fluid content (vitreous humor) 23 present in the eye 24 into the MN array 21 and simultaneously, desorption of the disposed drug 27 from the MN array 21′ to the vitreous humor 23 in the eye 24 via diffusion.

When the required amount of drug 27 has been released into the eye 24, a light source 26, as shown in FIG. 12C, is used to illuminate the swelled MN array 21′. Upon illumination with a desired bandwidth from light source 26, as prescribed by the photo-responsive configuration of the hydrogel composition, the swelled MN array 21 starts to deswell. FIG. 12D illustrates the easy removal of the deswelled MN array 21″ from the eye 24.

To convert hydrogel MN into a photo-responsive MN, as was briefly discussed above, hydrogel used is often first combined with photochromic dyes. Spiropyrans are a series of classic photochromic dyes which have been widely used in the development of stimuli-responsive materials. Other photochromic dyes used in the development of stimuli-responsive materials include naphthopyran and azobenzene. Often but not necessarily, spiropyran functionalized N-isopropylacrylamide (NIPPAM) hydrogels have been developed as a photo-responsive material and used in microfluidic applications as values and actuators. When the hydrogel is subject to light, the hydrogel switches a hydrophilic ring-open form to a hydrophobic ring-closed form because of the photochromic dyes. This hydrophobic nature leads to deswelling or shrinking of the hydrogel. This feature of hydrogel synthesized with photochromic dyes aids in reversing the MN adhesion by inducing de-swelling for easy extraction.

The embodiment herein encloses a fabrication process for a spiropyran conjugated hydrogel MN with three different compositions. As a non-limiting aspect of the embodiments herein, 0%, 10%, and 20% of spiropyran-conjugated poly (NIPPAM) gels (PNS) with polyvinyl alcohol (PVA) is used in fabricating the MN arrays. The swelling and deswelling kinetics, and the penetration force and adhesion strengths with and without light stimulation are compared and presented herein to demonstrate the performance of the photo-responsive hydrogel MNs.

### Method

### A. Material Preparation

The MNs presented herein often includes at least two material systems, one with the polymer (PVA herein) which provides the mechanical strength of the MN and the other with the photochromic dye (spiropyran conjugated PNS herein). Polyvinyl alcohol (PVA) hydrogel (Mw 146,000-186,000, 99% hydrolysis; Sigma Aldrich, St. Louis, MO, USA) is used with a concentration of 16% PVA to water w/w. The PVA and water mixture is stirred at 95° C. until the PVA is fully dissolved. Then the mixture is vacuumed and degassed before mixing with the spiropyran conjugated PNS. The spiropyran is prepared using an informed procedure known to those skilled in the art. ¹H-NMR of spiropyran is measured (400 MHz, CDCl₃): δ 7.17 (t, J=7.60 Hz, 1H), 7.06 (d, J=7.2 Hz, 1H), 6.83 (t, J=7.4 Hz, 1H),6.79 (d, J=10.2 Hz, 1H), 6.68-6.58 (m, 3H), 6.51 (d, J=7.7 Hz, 1H), 6.40 (d, J=17.3 Hz, 1H), 6.12 (dd, J=17.3, 10.5 Hz, 1H), 5.82 (d, J=10.4 Hz, 1H), 5.69 (d, J=10.2 Hz, 1H), 4.17 (t, J=6.7 Hz, 2H), 3.89 (t, J=6.4 Hz, 2H), 2.72 (s, 3H), 1.81-1.66 (m, 4H), 1.53-1.39 (m, 4H), 1.30 (s, 3H), 1.16 ppm (s, 3H). The NMR data measured herein matches reported data. PNS is prepared by polymerization of spiropyran and N-isopropylacrylamide (NIPAAM) in the present of azobisisobutyronitrile (AIBN), following the informed procedure. ¹H-NMR (400 MHz, DMF-d₇): δ 7.50 (br, 35H), 7.14 (dd, J=7.40 Hz, 3H), 7.05 (d, J=10.4 Hz, 1H) 6.88-6.84 (br, 1H), 6.80 (t, J=7.4 Hz, 1H), 6.75 (d, J=8.1 Hz, 1H), 6.58 (dd, J=13.4, 8.4 Hz, 2H), 5.83 (d, J=9.6 Hz, 1H), 3.96 (s, 45H), 2.27-2.16 (m, 40H), 1.81-1.38 (m, 80H), 1.13 ppm (s, 244H). These values match the reported data.

### B. Fabrication of MN Arrays

The fabrication process of the second example embodiment herein is the same as the fabrication process of the first example embodiment, as discussed above with respect to FIG. 9. In step 1, computer-aided design models of the MN array are created and converted into input files for a digital light processing based 3D printer (B9 Core 530, B9Creations, Rapid City, SD, USA) followed by printing a master mold (positive mold). The master mold of the MN array includes a group of MNs, wherein each respective MN has the preformed interlocking feature. SYLGARD 184 (Dow Corning, Midland, MI, USA) is used to cast soft fabrication molds (negative mold), following an informed procedure known to those skilled in the art. The elastomer used herein for casting is made of two parts: one is a curable silicone, and the other is a curing agent. The ratio used is 10:1 elastomer to curing agent, respectively. The elastomer is first mixed in a centrifuge unit (NuWind NU-C200R, NuAire, Plymouth, MN, USA) at 2300 rpm for 5 min to create a mixture. In step 2, the mixture is poured about the 3D-printed master mold to create the fabrication mold.

The hydrogel composition is different in the second embodiment as compared to the first embodiment. Herein, three hydrogel compositions (0%, 10%, and 20% of spiropyran-conjugated PNS with PVA) are synthesized to fabricate MN arrays. The hydrogel compositions are mixed by a magnetic stirrer. Finally, the mixture is cast over the fabrication mold, centrifuged in a centrifuge unit in step 3 and subjected to seven freeze-thaw cycles. In each cycle, the mixture is subjected to freezing for 8 hours at −20° C. and then thawing for 5 hours at 25° C.

### C. Experimental Setup and Procedures for Measurement of Adhesion and Penetration Forces

The experimental setup used to conduct the force vs distance measurement tests for the second example embodiment herein is same as that for the first example embodiment as shown in FIG. 11. The beneficial setup herein includes a linear motion system converted from a 3D printer (TAZ 6, LulzBot, Fargo, ND, USA) with a printhead carriage, a load cell (Miniature Low Profile Tension Link Load Cells 0.75 to 1″ Height, OMEGA, Norwalk, CT, USA), a data acquisition system (Model #USB-1608G, Measurement Computing, Norton, MA, USA), a light source (OmniCure S2000 Spot Curing System, Excelitas Technologies, Waltham, MA, USA). It is to be noted, however, that while the photochromic dye in the microneedle is most responsive to ultraviolet (UV) wavelength range of 100-400 nm, as known to those skilled in the art; the microneedle presented herein is capable of being responsive to visible light wavelength range of up to 700 nm.

The linear motion system was used to drive the MNs (it is to be noted that a single MN is shown in FIG. 11) into the polycaprolactone-based sclera-mimicking phantom, which is fabricated following standard procedures. The phantom is fixed on an acrylic holder. The load cell is used to measure the penetration and adhesion forces. Three MN arrays of different compositions used are as follows: 100% PVA, 90% PVA-10% PNS (w/w) and 80% PVA-20 PNS (w/w). The other components in the experimental setup include a computer and its CPU, and a power supply unit.

### D. Penetration Forces Measurement

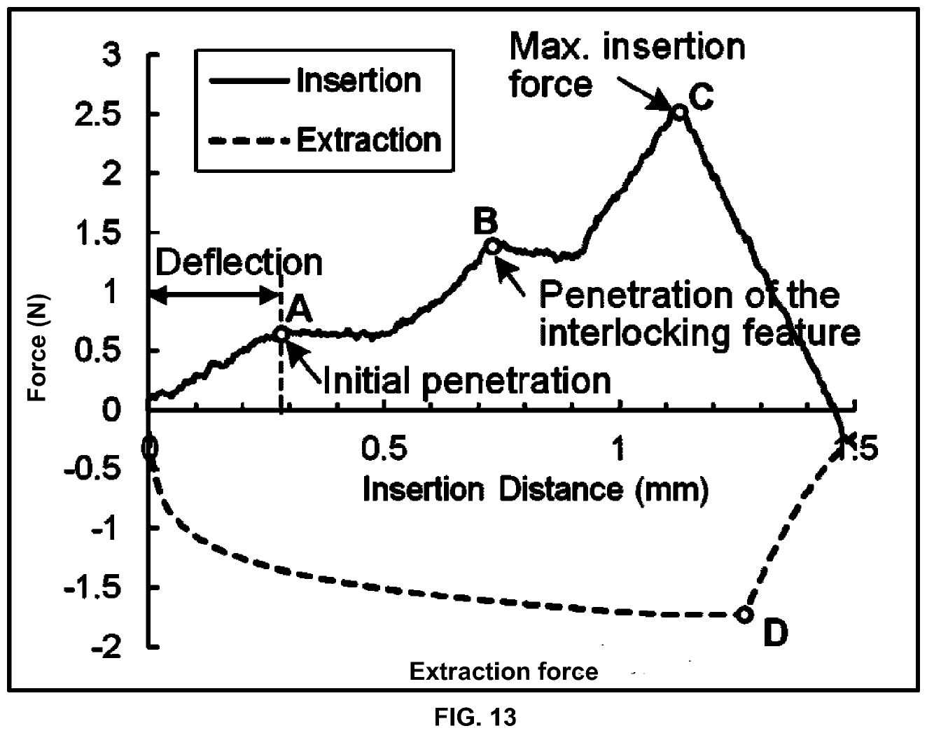

To measure the penetration force, the MN is lowered to right above the sclera-mimicking phantom 30 and then is programmed to advance 1.5 mm at a speed of 100 mm/min. An example of the recorded force is shown in FIG. 13, wherein until point A, it is seen that the MN deflected the phantom where the tip of MN cut through and penetrated into the phantom. At point B in FIG. 13, the preformed interlocking feature of the MN (array) disclosed herein penetrated into the phantom. The maximum recorded force at point C is used as the measure of the insertion force.

### E. Adhesion Forces Measurement

To measure the extraction force without light illumination, the inserted MNs continue to stay in the phantom for 10 minutes to allow swelling and then they are extracted by moving the MN holder up at a rate of 100 mm/min. An example of the recorded extraction force is also shown in FIG. 13. The force at point D is used as the extraction force for each measurement. In the cases of deswelling experiments, the MNs are allowed to swell for 10 minutes and then undergo light illumination using a light source. As was shown in FIG. 11, in the experimental setup herein, a UV light source was used to illuminate the MN (array) for 14 minutes at 10 mW/cm² irradiation of 365 nm before the MN (array) was extracted out of the phantom.

### F. Swelling and Deswelling Kinetics

To measure the swelling in the vitreous humor mimicking gel and deswelling under light illumination, an inverted microscope is used to take images of an MN and ImageJ is used to measure the change in area and the width of the preformed interlocking feature. The MNs are inserted into the vitreous humor mimicking gel contained in an acrylic box and sealed with a 1-mm layer of the sclera-mimicking phantom.

### G. Statistical Analysis

One-way analysis of variation (ANOVA) was also performed using EXCEL to examine the statistical significance of the differences of extraction and insertion forces among the 100% PVA, 90% PVA-10% PNS and 80% PVA-20 PNS MN arrays.

### Results

### A. Effect of PNS Composition on the Extraction Force

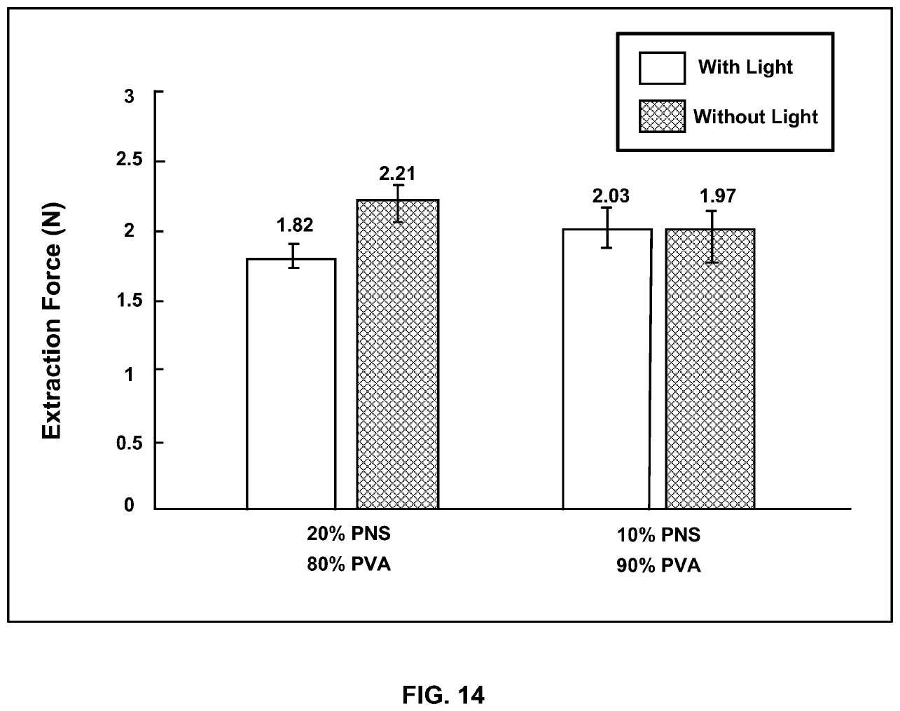

FIG. 14 shows the average results of ten extraction tests each on hydrogel composition of 80% PVA/20% PNS (w/w) and 90% PVA/10% PNS (w/w) MNs each with light illumination (non-hashed bars, as denoted in the legend) and without light illumination (hashed bars, as denoted in the legend) for deswelling. The extraction force is defined as the maximum measured force when the MNs inserted into the testing material are being pulled out from the sclera-mimicking phantom. The results show a significant difference between the extraction force with and without light illumination for the 80% PVA/20% PNS MNs.

### B. Effect of Light Illumination on the Extraction Force

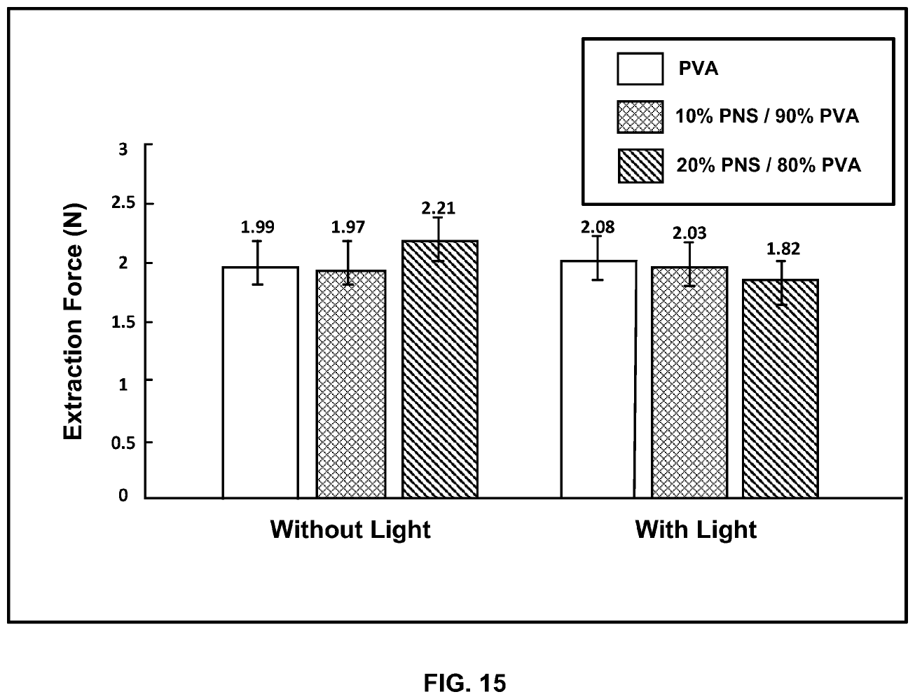

FIG. 15 shows a comparison of the extraction force in two groups, one with light illumination (as shown by the left 3 bars) and the other without light illumination (as shown by the right 3 bars). In each group, the results of three different MN compositions (100% PVA (non-shaded bars), 10% PNS/90% PVA (cross-hatched bars) and 20% PNS/80% PVA (diagonal-filled bars)) are shown. Without light illumination, the 20% PNS/80% PVA MNs show the highest extraction force, while with light illumination, the same MNs show the lowest extraction force, signaling easy extraction due to deswelling of the MNs. MNs of both 100% PVA and 10% PNS/90% PVA show insignificant change in extraction force with and without light illumination respectively. A UV light source is used herein for the light illumination, as the PNS is known by those skilled in the art to be most responsive to this wavelength. To protect the eye while applying the illumination, a white light frequency instead of a UV frequency helps avoid potential damage to the retina.

### C. Effect of PNS Composition on Insertion Force

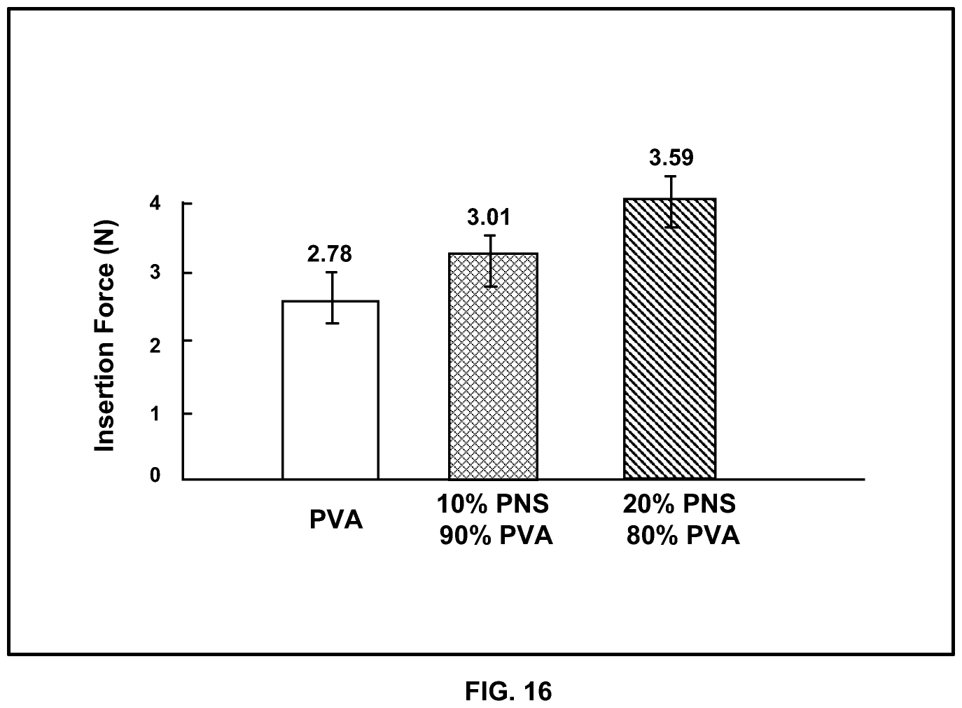

FIG. 16 compares the insertion forces of 10 averaged result for three MN compositions; 100% PVA, 10% PNS 90% PVA, 20% PNS 80% PVA. The insertion force is defined as the maximum recorded force when a MN is inserted into the test material while no light illumination is introduced. The results show that the insertion force has a slight increase as the composition of PNS increases moving from leftmost bar to the rightmost bar. This could be due to the weakening of the mechanical properties of the hydrogel with the addition of PNS which is not as strong as PVA. With lower mechanical properties, the MNs do not cut through the tissue easily and are more susceptible to buckling or bending while penetrating the tissue. While it is expected that with a higher fraction of spiropyran infused hydrogel, a better deswelling behavior can be achieved, but this benefit comes with the tradeoff of a minor increase in insertion force, yet the penetration force is still within acceptable limits.

### D. Swelling and Deswelling Kinetics

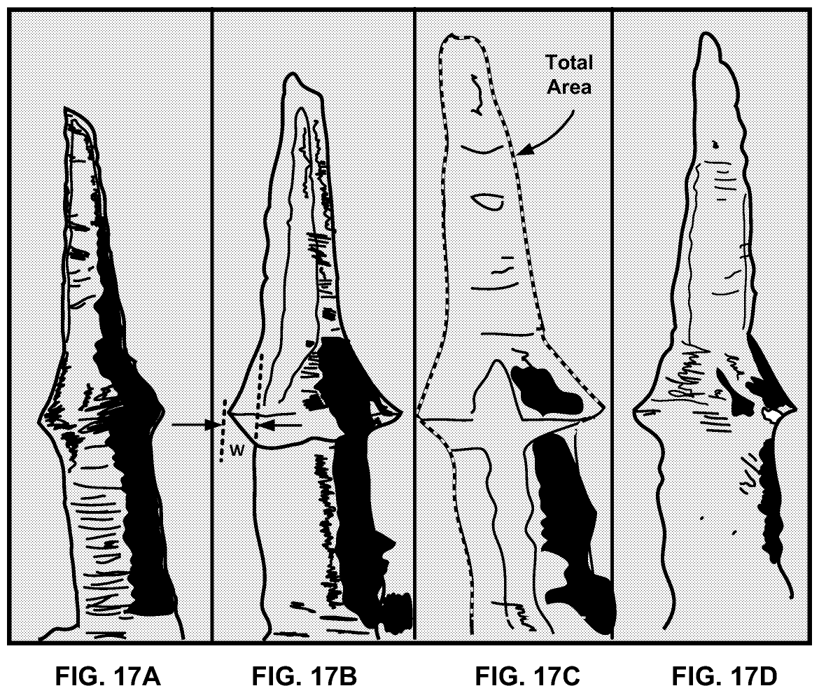

FIG. 17A through FIG. 17D is a graphical representation of the real image showing the swelling and deswelling of a MN (20% PNS/80% PVA) over time. The MN is submerged into a vitreous-mimicking fluid. FIG. 17A shows the MN in its initial state at time T=0 min. The swelling of MN is observed, and it is seen at time T=4 min in FIG. 17B, the width (denoted as w) of the preformed interlocking feature was increased. The swelling continues and it is observed that at time T=10 min in FIG. 17C the total area (marked by dashed lines) of the MN is seen to be increased significantly. Light illumination is applied to the MN at the 10 minutes mark for 14 minutes. Surprisingly and unexpectedly, the MN observed at time T=24 min in FIG. 17D was observed to have a de-swollen geometry with decreased width and decreased area as compared to swollen width and swollen area of FIG. 17C.

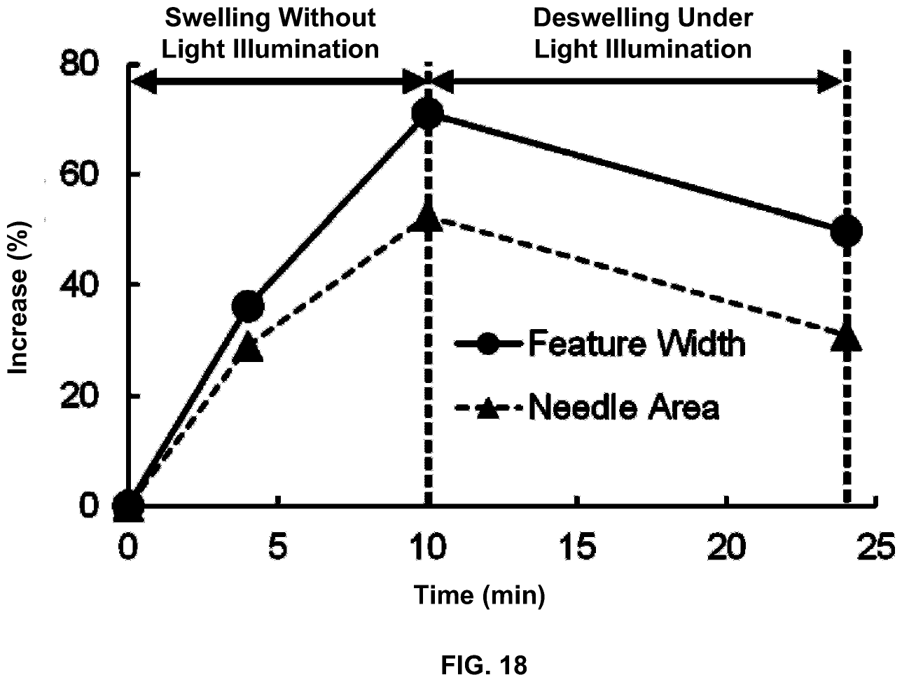

The width of the preformed interlocking feature and the total area of the MN were used to quantify the swelling/deswelling kinetics, as shown in FIG. 18. At the 10 minute mark, the width of the preformed interlocking feature and the total area of the MN increased by 70% and 50%, respectively. Upon light illumination, often but not limited to, about 20% reduction of MN in both the width and total area is observed. No significant reduction is observed by continuing to illuminate the MNs. This deswelling of MN contributes to the reduction of adhesion force, as shown in FIG. 13. As shown in FIG. 14, a higher concentration of PNS leads to a more significant reduction in extraction force. However, increasing the PNS content also leads to an increase in insertion force, as shown in FIG. 16.

It should be emphasized that the above-described embodiments and the specific examples of the present invention, particularly, any “preferred” embodiments, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

I/We claim:

1. A microneedle array, comprising:
a plurality of microneedles, wherein each of a respective microneedle of the plurality of microneedles further comprises:
a base;
a needle tip;
an elongated structure therebetween the base and the needle tip; and
a preformed interlocking feature configured about the elongated structure wherein the preformed interlocking feature is arranged with a width that decreases in the needle tip direction and decreases in the base direction.

2. The microneedle array of claim 1, wherein
the width of the preformed interlocking feature increases in a first exist angle adjacent the needle tip direction and increases in a second exist angle adjacent the base direction.

3. The microneedle array of claim 2, wherein the first exist angle is at least 20 degrees.

4. The microneedle array of claim 2, wherein the second exist angle is at least 20 degrees.

5. The microneedle array of claim 1, wherein
the preformed interlocking feature is arranged to increase in width between 5 to 300 percent upon a fluid tissue contact.

6. The microneedle array of claim 5, wherein
the preformed interlocking feature is arranged to decrease in width by 10 percent down to its original width prior to the fluid tissue contact upon illumination with light.

7. The microneedle array of claim 1, wherein
each of the plurality of microneedles comprises a hydrogel.

8. The microneedle array of claim 7, wherein
each of the plurality of microneedles comprises a photo-responsive compound coupled to the hydrogel.

9. The microneedle array of claim 1, wherein
the preformed interlocking feature of each of the plurality of microneedles is constructed from a photo-responsive compound coupled to a hydrogel, and wherein the remainder of each of the plurality of microneedles are configured from a non-photo-responsive hydrogel.

10. The microneedle array of claim 1, wherein each of the microneedles are configured from at least one of a natural polymer and a synthetic polymer.

11. The microneedle array of claim 10, wherein the natural polymer further comprises a polymer selected from a protein or a polysaccharide.

12. The microneedle array of claim 10, wherein the synthetic polymer further comprises a polymer selected from a polyvinyl alcohol (PVA), a polyethylene glycol (PEG), or a polyacrylic acid (PAA).

13. The microneedle array of claim 8, wherein the photo-responsive compound is selected from a spiropyran-conjugated N-isopropylacrylamide, a naphthopyran or an azobenzene.

14. A method of drug release, comprising:
inserting at least one or more microneedles each into a target location wherein each of the one or more microneedles has a configured preformed interlocking feature;
increasing the width of the preformed interlocking feature upon a fluid tissue contact so as to secure each of the one or more microneedles at the target location;
releasing a self-contained drug disposed in each of the one or more microneedles; and
illuminating the one or more microneedles using a light source, wherein the width of the preformed interlocking feature decreases so as to enable the extraction of the one or more microneedles from the target location.

15. The method of drug release of claim 14, further comprising:
configuring the preformed interlocking feature to increase in width between 5 and 300 percent upon the fluid tissue contact.

16. The method of drug release of claim 15, further comprising:
configuring the preformed interlocking feature to decrease in width by 10 percent down to its original width prior to the fluid tissue contact upon illumination with light.

17. The method of drug release of claim 14, further comprising:
utilizing a hydrogel to configure each of the one or more microneedles.

18. The method of drug release of claim 17, further comprising:
coupling a photo-responsive compound to the hydrogel to configure each of the one or more microneedles.

19. The method of drug release of claim 14, further comprising:
utilizing a photo-responsive compound coupled to a hydrogel for constructing the preformed interlocking feature of each of the one or more microneedles and utilizing a non-photo-responsive hydrogel for constructing the remainder of the one or more microneedles.

20. The method of drug release of claim 14, further comprising:
utilizing at least one of a natural polymer or a synthetic polymer for constructing the one or more microneedles.

21. The method of drug release of claim 20, further comprising:
a natural polymer selected from a protein or a polysaccharide.

22. The method of drug release of claim 20, further comprising:
a synthetic polymer selected from a polyvinyl alcohol (PVA), a polyethylene glycol (PEG), or a polyacrylic acid (PAA).

23. The method of drug release of claim 18, further comprising:
utilizing the photo-responsive compound selected from a spiropyran-conjugated N-isopropylacrylamide, a naphthopyran or an azobenzene.

24. A microneedle array fabrication process, comprising:
molding a microneedle array, wherein a respective microneedle of the microneedle array has a configured preformed interlocking feature;
casting a fabrication mold of the microneedle array;
disposing a hydrogel concentration into the fabrication mold;
centrifuging the fabrication mold; and
subjecting the fabrication mold having the disposed hydrogel concentration with one or more freeze-thaw cycles.

25. The microneedle array fabrication process of claim 24, wherein
the step of molding utilizes a three-dimensional printer.

26. The microneedle array fabrication process of claim 24, wherein
the step of disposing includes coupling a photo-responsive compound to the hydrogel concentration.
